(19) United States
(12) Patent Application Publication
(10) Pub. No.: US 2024/0101749 A1
(43) Pub. Date: Mar. 28, 2024

(54) RESIN COMPOSITION

(71) Applicant: 4D MEDICINE LIMITED, Birmingham (GB)

(72) Inventors: Andrew Peter DOVE, Birmingham, West Midlands (GB); Andrew Christopher WEEMS, Birmingham, West Midlands (GB)

(21) Appl. No.: 18/263,327

(22) PCT Filed: Jan. 28, 2022
(86) PCT No.: PCT/GB2022/050237

§ 371 Date: Jul. 27, 2023

(30) Foreign Application Priority Data
Jan. 28, 2021 (GB) 2101142.4

(51) Int. Cl. C08G 18/44; A61L 27/18; C08G 18/10; C08G 18/38

(52) U.S. Cl. CPC C08G 18/44; A61L 27/18; C08G 18/10; C08G 18/3848

(57) ABSTRACT

A resin composition comprises a prepolymer, a reactive diluent, and optionally a crosslinker, wherein the reactive diluent has an absorption maximum (Amax) or excitation maximum (Amax) of between 350 and 520 nm for example in acetone and at pH 7.

This invention relates generally to resin compositions for producing polymers. More specifically, although not exclusively, this invention relates to resin compositions for producing biocompatible cross-linked polymers, methods of producing the same, and methods of crosslinking the same, for example using stereolithography.

There is a clear need for biocompatible materials for use in the body, for example, for use in fabricating medical devices such as implants. Those biocompatible materials preferably have non-toxic degradation properties or products when located within the body (for example, non-acidic degradation products). There is a further need for biocompatible materials which have tunable physical or mechanical properties.

One such tunable property that is desirable in biocompatible materials is shape memory. Shape memory polymers (SMPs) are a class of programmable, stimuli-responsive materials that exhibit shape-changing transformations in response to exposure to external stimuli. Objects fabricated from SMPs are formed in an original primary shape which may be deformed into a metastable secondary shape, which, upon exposure to an appropriate stimulus, revert to the original primary shape. Examples of appropriate stimuli include heating (direct or indirect), for example Joule heating, radiation and laser heating, microwaves, pressure, moisture, the presence or absence of solvent or solvent vapours, and/or change in pH.

SMPs have been considered for use in a variety of applications not limited to medicine, including for example aeronautics, textiles, and automotive applications. SMPs have been proposed or used, for example, in self-healing systems, self-deployable structures, actuators and sensors.

It is of particular interest to use SMPs in biomedical applications, for example, in the fabrication of stimuli-responsive biomaterials and/or medical devices, e.g. implantable medical devices such as tissue scaffolds. This is particularly interesting because an implantable medical device may be produced in a desired ‘original’ shape, which is subsequently deformed into a minimally invasive (or at least ‘less invasive’) secondary shape that is deliverable with minimal (or less) trauma to the patient. Once the device, e.g. a tissue scaffold or stent, is in place, a stimulus such as body heat, causes the device to revert to its ‘original’ primary shape, e.g. expanding to a desired shape to perform its function and/or to exert force on the surrounding tissue.

In the fabrication of tissue scaffolds, it has been suggested that homogeneity of pore size, and/or homogeneous morphology, is an important factor for successful outcomes.

Traditional manufacturing techniques proposed for fabricating scaffolds with high porosities include templating, electrospinning and foaming. However, one major limitation with these techniques is that morphological heterogeneity is observed across the scaffold material. Additionally, scaffolds fabricated using these techniques may have fewer interconnected pores after processing than theorized. Ideal pore sizes have been found to range from between 100 to 700 μm. It would be advantageous for the pore connectivity and size to be tunable for specific applications. For example, pore sizes of 100 μm will promote chondrogenesis (the process by which cartilage is formed from condensed mesenchyme tissue), whereas pore sizes of 400 to 500 μm will promote osteogenesis (the formation of bone) without initial cartilage formation and vascularisation. Pore size has also been shown to be important to allow control over biomolecules such as proteins found in healing processes, e.g. osterocalcin, osteopontin, collagen, bone morphogenetic protein, and bone sialoprotein mRNA expressions. In addition, it has been shown that a specific pore size is important in each of the rate of bone formation, cell proliferation rates, and production of different tissue types. The ability to control the connectivity of pores within porous scaffolds has also been shown to be significant in the control of cellular scaffold mineralisation, resulting in faster bone growth. In contrast, uncontrolled or untailored pore design can result in non-native tissue infiltration, such as connective tissue forming instead of bone.

Therefore, in the production of medical devices and biomaterials such as tissue scaffolds, it would be highly advantageous to be able to achieve precise scaffold shapes (geometries) and properties, by controlling pore size, homogeneity, and interconnectivity, for specific applications. This has the potential to lead to a reduction in healing time for an improved quality of life.

In our patent application WO2018/229456A1, we describe an advantageous resin composition that meets at least some of these objectives. The resin composition comprises a prepolymer and optionally one or more diluent(s), the prepolymer comprising repeating units having at least one carbonate linkage and at least one unsaturated side-chain, the at least one optional diluent(s) comprising at least one unsaturated side-chain, wherein either or both of the prepolymer and the at least one optional diluent(s) comprises at least one O═C—N linkage, preferably a urethane linkage. These resin compositions may be used to form 3D-printed objects comprising a cross-linked polymer with advantageous properties over prior art polymers, e.g. biocompatibility, homogeneity of pore size, degradability into non-toxic products, tunability of mechanical properties, and the ability to functionalise the surface of the 3D-printed object post-polymerisation.

One of the challenges associated with 3D printing of resin compositions is the problem of over-curing caused by uncontrolled polymerisation and heating. This leads to loss of precision of surface features on the 3D printed object. Hardware approaches, for example modification of the printer set-up, have been proposed to overcome this problem (e.g. as reported in Sci. Adv.; 2017, 3 (12); eaao5496). An alternative approach is to include additional agents in the resin composition. A variety of different approaches have been taken to prevent over-curing by including an additional agent in the resin composition, including utilizing [6,6]-phenyl-C61-butyric acid methyl ester (PCBM) molecule as the cationic photoinitiator in combination with an oxidizing agent, AgPF₆, to trigger the polymerization of the photoresist (J. Phys. Chem. C.; 2017, 121, 31, 16970-16977) and the use of radical quenchers (Macromolecular Research volume 14, pp 559-564 (2006)).

The use of photoinhibition agents in resin compositions to produce a crosslinked polymer object has also been proposed. The purpose of the photoinhibitor is to gain control over the cure depth by absorbing light in the region (e.g. the narrow-band light source) employed by a stereolithography apparatus. This achieves greater resolution of more intricate features in the resulting crosslinked polymer object. Clearly this is advantageous when producing, for example, patient-specific implants for use in tissue engineering therapies.

The article Biomater. Sci., 2014, 2, 472-475 entitled “A microstereolithography resin based on thiol-ene chemistry: towards biocompatible 3D extracellular constructs for tissue engineering” describes the use of Kalsec Durabrite® Oleoresin Paprika Extract NS (a common food additive) as a photoinhibitor in a thiol-ene based resin composition.

Resin compositions (e.g. those based on thiol-ene chemistry) often contain multiple reagents. For example, the resin compositions of WO2018/229456A1 may contain a prepolymer, one or more reactive diluents, a crosslinker, and/or a viscosity modifier. Although not essential, a photoinhibitor may be used in these thiol-ene based compositions to enhance resolution of intricate features. However, this adds an additional reagent to the composition, which increases complexity during manufacture. Moreover, the use of further species may add complications with regards to degradation products and/or in situ toxicity (especially when being used in vivo). Further the use of further species may be detrimental to the properties (e.g. structural properties) of the resulting polymer. It would therefore be advantageous to be able to enhance printing accuracy, whilst simplifying the resin composition for use in producing a 3D printed object.

Accordingly, a first aspect of the invention provides a resin composition, the resin composition comprising a prepolymer, a reactive diluent, and optionally a crosslinker, wherein the reactive diluent has an absorption maximum (λₘₐₓ) between 350 and 520 nm, for example, when dissolved in a solvent (e.g. acetone and at pH 7).

The resin composition of the invention is curable, e.g. photocurable.

Advantageously, the reactive diluent having an absorption maximum (λₘₐₓ) of between 350 and 520 nm is able to act as a photoinhibitor in the resin composition without the need for addition of a further reagent. Therefore, the resin composition is usable to retain spatial control during the printing process to print more complex geometries in contrast to reactive diluents that do not exhibit this property.

More advantageously, the reactive diluent is incorporated into the crosslinked polymer structure, which means that there is no need for an additional agent, which remains free within the polymer matrix, and may lead to leaching of potentially toxic, unbound species from the crosslinked polymer.

By a reactive diluent, we mean a compound that is able to react with the prepolymer. That is, the reactive diluent contains a functional group that is capable of reacting with a functional group on the prepolymer and/or the crosslinker to result in a chemical bond, e.g. an ionic bond or a covalent bond, preferably a covalent bond. The reactive diluent may be usable to crosslink the prepolymer to form a crosslinked network in the resulting object (e.g. 3D printed object). Thus, it will be understood that the reactive diluent is a different compound to the prepolymer.

In embodiments, the reactive diluent is not a polymer or copolymer.

By absorption maximum, we mean the wavelength at which the reactive diluent shows maximum absorbance (e.g. using a UV/Vis spectrophotometer) and/or the wavelength at which the reactive diluent shows maximum excitation (e.g. using a fluorimeter). In this specification, the terms “absorption maximum” and “excitation maximum” are used interchangeably.

In embodiments, the reactive diluent has a relative molecular mass (Mᵣ) of less than 1000. In embodiments, the reactive diluent has a relative molecular mass (Mᵣ) of less than 900, less than 800, less than 700, less than 600 or less than 500.

In embodiments, the reactive diluent has a relative molecular mass (Mᵣ) of at least 200, at least 250, at least 300, at least 350 or at least 400.

In embodiments, the reactive diluent may comprise one or more unsaturated groups, e.g. one or more -ene group(s) (e.g. one or more alkene group(s)) and/or one or more alkyne groups, that is capable of reacting with a functional group on the prepolymer and/or the crosslinker. The one or more unsaturated groups, e.g. one or more -ene group(s) and/or the one or more alkyne groups, may be located in-chain or as a pendant group. The at least one, some, or all of the one or more -ene group(s) may be an allyl group.

In embodiments, the reactive diluent may comprise at least one unsaturated side chain. In embodiments, the unsaturated side-chain of the reactive diluent may comprise an aliphatic moiety (e.g. an alkene, an alkyne), or an aromatic moiety, for example, a phenyl group or a substituted phenyl group, a heterocyclic aromatic moiety, or a polycyclic aromatic hydrocarbon. The unsaturated side-chain may be linear or may be cyclic.

In embodiments, the reactive diluent may comprise one or more (e.g. two, three or four) unsaturated aliphatic C—C bonds, for example alkyne or allyl groups, for example, the diluent may comprise two allyl groups, or three allyl groups, or four allyl groups.

The reactive diluent may comprise the general formula (i):

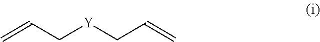

wherein Y comprises an alkyl and/or an aryl moiety, or a functionalised alkyl and/or a functionalised aryl moiety.

In embodiments the reactive diluent may have the general formula (ia):

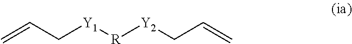

Where Y1 and Y2 may be the same or different and one or both may comprise a urethane or urea moiety, for example one or both of Y1 and Y2 may comprise a cyclic urea or urethane moiety, and R may be selected from an alkyl, substituted alkyl, aryl or substituted aryl group, for example an aliphatic or aromatic aryl or substituted aryl group.

Additionally or alternatively, the reactive diluent may comprise one or more thiol group(s), i.e. an —S—H group, that is capable of reacting with a functional group on the prepolymer and/or the or a crosslinker (e.g. an -ene group or an alkyne group).

An important consideration for biological applications of 3D printed objects is the toxicity of the starting materials. It is known that acrylates exhibit greater toxicity compared with allyl groups. Advantageously, the resin composition according to the invention wherein the functional groups are allyl groups has greater biocompatibility than those resin compositions of the prior art containing acrylate groups.

The absorption maximum (λₘₐₓ) of the reactive diluent lies between 350 and 520 nm in a solvent (e.g. acetone) at pH 7. In embodiments, the absorption maximum (λₘₐₓ) of the reactive diluent may lie between any one of 350, 360, 370, 380, 390, 400, 410, 420, 430, 440, 450, 460, 470, 480, 490, 500, 510 nm to any one of 520, 510, 500, 490, 480, 470, 460, 450, 440, 430, 420, 410, 400, 390, 380, 370, 360 nm in a solvent (e.g. acetone) at pH 7.

In embodiments, the reactive diluent may comprise a —O═C—N— linkage, for example, one or more of a urethane (i.e. carbamate) linkage and/or a urea (i.e. carbamide) linkage. We prefer the reactive diluent to comprise at least one urea linkage (e.g. one, two or more urea linkages). In embodiments, the reactive diluent may comprise a moiety comprising a urea (or urethane) linkage in a cyclic moiety, i.e. a cyclic urea (or urethane). The reactive diluent may comprise more than one moiety (e.g. two or more moieties) containing a urea linkage in a cyclic moiety, i.e. a cyclic urea. The cyclic moiety containing the urea linkage may be a five- or six-membered cyclic moiety. In embodiments wherein the reactive diluent has the general formula (i) (or (ia)), the moiety “Y” (or Y₁ and/or Y₂) may comprise one or more cyclic moieties containing a urea linkage, e.g. one or more (e.g. two) six membered cyclic moieties wherein one or more or each contain at least one (i.e. one, two or more) urea linkage, preferably one.

In some embodiments wherein the reactive diluent has the general formula (i), the moiety “Y” (or Y₁ and/or Y₂) comprises one or more (e.g. two) cyclic moieties of formula (iii):

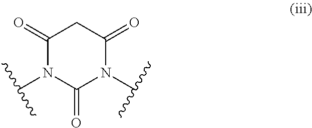

In embodiments, the reactive diluent may be derived from a reaction product of an isocyanate (preferably a diisocyanate (or higher)), e.g. isophorone diisocyanate, with an amine (e.g. allylamine). In embodiments, the reactive diluent may be derived from reaction of an intermediate product with malonyl chloride, wherein the intermediate product is formed by reaction of an isocyanate (preferably a diisocyanate (or higher)), for example isophorone diisocyanate with an amine (e.g. allylamine).

In embodiments, the reactive diluent has the formula (ii):

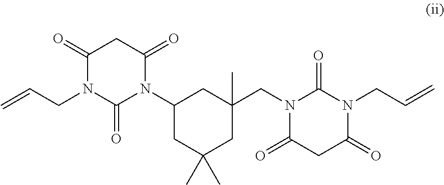 [SMILES: C=CCN1C(=O)CC(=O)N(CC2(C)CC(N3C(=O)CC(=O)N(CC=C)C3=O)CC(C)(C)C2)C1=O]

It has been surprisingly found that a reactive diluent having the formula (ii) is capable of functioning as a reactive photoinihibitor over at least a proportion of the 350 nm to 520 nm light range. This allows for more complex parts to be printed without requiring additional resin components, which may not be soluble or could otherwise alter the resin composition or be otherwise deleterious.

In embodiments, the resin composition may comprise a further diluent or diluent(s), for example two diluents, three diluents, four diluents, or more than four diluents. The further diluent or diluent(s) may be reactive diluents or unreactive diluents. In embodiments, one or more or each of the further diluent(s) may comprise at least one unsaturated side-chains, preferably plural unsaturated side chains.

In embodiments, the resin composition may comprise viscosity modifiers, for example, one or more non-reactive viscosity modifiers. An example of a suitable viscosity modifier is propylene carbonate.

In embodiments, the prepolymer may comprise repeating units, wherein each repeating unit has at least one carbonate linkage. In embodiments, the prepolymer may be a polycarbonate.

In embodiments, the prepolymer may comprise repeating unit, wherein each repeating unit has a functional group that is capable of reacting with a functional group on the reactive diluent and/or the crosslinker. In embodiments, the functional group is an -ene group (e.g. an unsaturated side-chain). In embodiments, the functional group is an alkyne group.

In embodiments, the prepolymer may comprise one or more unsaturated groups, for example one or more -ene group(s), e.g. one or more alkene group(s), or one or more alkyne groups, that is capable of reacting with a functional group on the reactive diluent and/or the crosslinker. The one or more unsaturated groups, e.g. one or more -ene group(s) or alkyne group(s), may be located in-chain or as a pendant group. The at least one, some, or all of the one or more -ene group(s) may be an allyl group. In embodiments, the prepolymer may comprise at least one unsaturated side chain.

The prepolymer may comprise repeating units, wherein each repeating unit has at least one carbonate linkage and at least one unsaturated side-chain.

In embodiments, the unsaturated side-chain of the prepolymer may comprise an aliphatic moiety (e.g. an alkene, an alkyne), or an aromatic moiety, for example, a phenyl group or a substituted phenyl group, a heterocyclic aromatic moiety, or a polycyclic aromatic hydrocarbon. The unsaturated side-chain may be linear or may be cyclic.

In embodiments, the prepolymer may comprise the formula (iii):

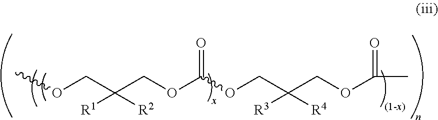

wherein R¹ is a hydrogen atom or an aliphatic or an aromatic moiety or group, R² is a hydrogen atom or an aliphatic or an aromatic moiety or group, R³ is a hydrogen atom or an aliphatic or an aromatic moiety or group, and R⁴ is a hydrogen atom or an aliphatic or an aromatic moiety or group, and wherein n is a number that is less than one hundred, e.g. 99, 98, 97, 96, 95, 94, 93, 92, 91, 90, 80, 70, 60, 50, 40, 30, 20, or 10, and x is a number between 0 and 1.

In embodiments, any or all of R¹, R², R³ and/or R⁴ may be a hydrogen atom, an alkyl chain, e.g. methyl, ethyl, propyl, butyl and so on, and isomers thereof; an aromatic ring, an aliphatic ring, an allyl ether, an acrylate (e.g. with modification), and/or an allyl ester.

In embodiments wherein one or more or each of R¹, R², R³, and/or R⁴ is an aromatic group, the aromatic group may be one of, or a combination of, an aromatic hydrocarbon group, and/or an aromatic heterocyclic group.

In embodiments, the prepolymer may comprise a —O═C—N— linkage, for example, one or more of a urethane (i.e. carbamate) linkage and/or a urea (i.e. carbamide) linkage. In embodiments, the prepolymer may be chain extended using an isocyanate compound to create a urethane linkage. The isocyanate compound may be aliphatic or aromatic. The isocyanate compound typically comprises two or more isocyanate moieties. For example, the isocyanate may be isophorone diisocyanate (IPDI). In alternative embodiments, the isocyanate is hexamethylene diisocyanate (HDI). However, any suitable diisocyanate may be used, e.g. tetramethylxylene diisocyanate (TMXDI), phenylene diisocyanate, toluene diisocyante (TDI), xylylene diisocyanate (XDI), cyclohexylene diisocyanate and so on.

In embodiments, the prepolymer may comprise the formula (iv):

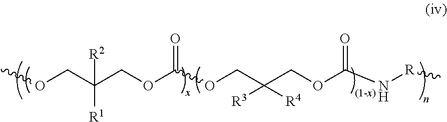

wherein R group is an aliphatic or an aromatic moiety or group, R¹ is a hydrogen atom or an aliphatic or an aromatic moiety or group, R² is a hydrogen atom or an aliphatic or an aromatic moiety or group, R³ is a hydrogen atom or an aliphatic or an aromatic moiety or group, and R⁴ is a hydrogen atom or an aliphatic or an aromatic moiety or group, and wherein n is a number that is less than one hundred, e.g. 99, 98, 97, 96, 95, 94, 93, 92, 91, 90, 80, 70, 60, 50, 40, 30, 20, or 10, and x is a number between 0 and 1.

In embodiments, the prepolymer may comprise the formula (v):

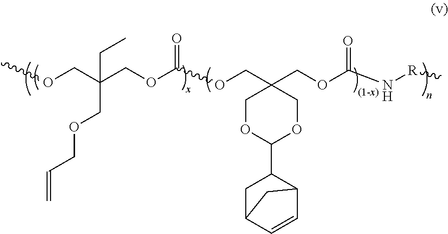

wherein the R group is an aliphatic or an aromatic moiety or group, and wherein n is a number that is less than one hundred, e.g. 99, 98, 97, 96, 95, 94, 93, 92, 91, 90, 80, 70, 60, 50, 40, 30, 20, or 10, and x is a number between 0 and 1.

In an embodiment, R is an alkyl group comprising six carbons.

In embodiments, the prepolymer may be fabricated from components comprising the formulae (vi) and a diisocyanate (I):

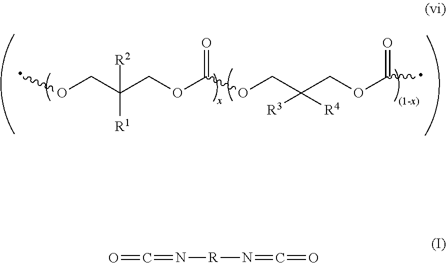

wherein R group is an aliphatic or an aromatic moiety or group, R¹ is a hydrogen atom or an aliphatic or an aromatic moiety or group, R² is a hydrogen atom or an aliphatic or an aromatic moiety or group, R³ is a hydrogen atom or an aliphatic or an aromatic moiety or group, and R⁴ is a hydrogen atom or an aliphatic or an aromatic moiety or group, and wherein n is a number that is less than one hundred, e.g. 99, 98, 97, 96, 95, 94, 93, 92, 91, 90, 80, 70, 60, 50, 40, 30, 20, or 10, and x is a number between 0 and 1.

In alternative embodiments, the prepolymer does not contain an O═C—N linkage (e.g. a carbamate (e.g. urethane) or carbamide (e.g. urea) linkage).

In embodiments wherein one or more or each of R, R¹, R², R³, and/or R⁴ is or comprises an aromatic hydrocarbon group, the aromatic hydrocarbon group may comprise one of, or a combination of, a phenyl ring and/or a substituted phenyl ring. There may be one, two, three, four, or five additional substituents on the phenyl ring. The substituents are bonded directly to the phenyl ring, and may be one of, or a combination of, fluorine, chlorine, bromine, iodine, a hydroxyl group, an amine group, a nitro group, an alkoxy group, a carboxylic acid, an amide, a cyano group, a trifluoromethyl, an ester, an alkene an alkyne, an azide, an azo, an isocyanate, a ketone, an aldehyde, an alkyl group consisting of a hydrocarbon chain, or a hydrocarbon ring, an alkyl group consisting of other heteroatoms such as fluorine, chlorine, bromine, iodine, oxygen, nitrogen, and/or sulphur. The alkyl group may comprise a hydroxyl group, an amine group, a nitro group, an ether group, a carboxylic acid, an amide, a cyano group, trifluoromethyl, an ester, an alkene an alkyne, an azide, an azo, an isocyanate, a ketone, an aldehyde, for example. The substituents may be another aromatic group, for example, R, R¹, R², R³, and/or R⁴ may comprise a phenyl substituted with a further phenyl ring. In embodiments, the R, R¹, R², R³, and/or R⁴ group may be a phenyl ring, substituted with a second phenyl ring, which in turn is substituted with a third phenyl ring.

In embodiments wherein one, more or each of R, R¹, R², R³, and/or R⁴ is an aromatic group, the aromatic group may be a polycyclic aromatic hydrocarbon, for example, naphthalene, anthracene, phenanthrene, tetracene, chrysene, triphenylene, pyrene, pentacene, benzo[a]pyrene, corannulene, benzo[ghi]perylene, coronene, ovalene, fullerene, and/or benzo[c]fluorene. The R group may be bonded to the triphenylene derivative by any isomer of the polycyclic aromatic hydrocarbons described, for example, 1-napthalene, 2-napthalene, 2-anthracene, 9-anthracene. The polycyclic aromatic hydrocarbon group may be substituted with other moieties such as aryl groups, alkyl groups, heteroatoms, and/or other electron withdrawing or electron donating groups.

In embodiments wherein one, more or each of R, R¹, R², R³, and/or R⁴ is an aromatic heterocyclic group, the heterocyclic group may be a four membered ring, a five membered ring, a six membered ring, a seven membered ring, an eight membered ring, a nine membered ring, a ten membered ring, or a fused ring. In embodiments, the heterocyclic group may be furan, benzofuran, isobenzofuran, pyrrole, indole, isoindole, thiophene, benzothiophene, benzo[c]thiophene, imidazole, benzimidazole, purine, pyrazole, indazole, oxazole, benzoxazole, isoxazole, benzisoxazole, thiazole, benzothiazole, pyridine, quinoline, isoquinoline, pyrazine, quinoxaline, acridine, pyrimidine, quinozoline, pyridazine, cinnoline, phthalazine, 1,2,3-triazine, 1,2,4-triazine, 1,3,5-triazine. pyridine or thiophene.

In embodiments wherein one, more or each of R, R¹, R², R³, and/or R⁴ is an aliphatic group, the aliphatic group may be one of, or a combination of, an n-alkyl chain, a branched alkyl chain, an alkyl chain comprising unsaturated moieties, an alkyl chain comprising heteroatoms, for example, fluorine, chlorine, bromine, iodine, oxygen, sulphur, nitrogen. The alkyl chain may comprise unsaturated portions, comprising alkenes, or aromatic moieties. The alkyl chain may comprise functional groups for further derivatisation of the triphenylene derivative. For example, the functional groups may be one or more of an azide, a carbonyl group, an alcohol, a halogen, or an alkene.

R, R¹, R², R³, and/or R⁴ may comprise an aliphatic ring, or an aromatic ring. R, R¹, R², R³, and/or R⁴ may comprise an allyl ether, an acrylate, a modified acrylate, and/or an allyl ester.

R, R¹, R², R³, and/or R⁴ may comprise a spirocyclic aliphatic ring, and/or a bridged ring, e.g. a norbornene ring.

We prefer R to be an aliphatic moiety.

The prepolymer may be a homopolymer of 5-[(allyloxy)methyl]-5-ethyl-1,3-dioxan-2-one. Additionally or alternatively, the prepolymer may be a homopolymer of 9-(5-norbornen-2-yl)-2,4,8,10-tetraoxa-3-spiro[5.5]undecanone. The prepolymer may comprise a copolymer of 5-[(allyloxy)methyl]-5-ethyl-1,3-dioxan-2-one and 9-(5-norbornen-2-yl)-2,4,8,10-tetraoxa-3-spiro[5.5]undecanone.

Additionally or alternatively, the prepolymer may comprise one or more thiol group(s), e.g. an —S—H group, that is capable of reacting with a functional group on the reactive diluent and/or the crosslinker (e.g. an -ene group or an alkyne group).

In embodiments, the crosslinker may comprise a functional group (e.g. at least one functional group, for example, 2, 3, 4, or more functional groups) that is capable of reacting with a functional group on the prepolymer and/or reactive diluent.

In embodiments, the crosslinker may comprise a thiol group (e.g. at least one thiol group, for example, 2, 3, 4, or more thiol groups) that is capable of reacting with a functional group on the prepolymer and/or reactive diluent. Additionally or alternatively, the crosslinker may comprise at least one -ene group (e.g. at least one alkene group, e.g. 2, 3, 4, or more -ene groups, for example one or more akene groups) that is capable of reacting with a functional group on the prepolymer and/or reactive diluent. Additionally or alternatively, the crosslinker may comprise at least one alkyne group that is capable of reacting with a functional group on the prepolymer and/or reactive diluent.

In embodiments, the crosslinker comprises one or more thiol moieties, for example, one thiol moiety, two thiol moieties, three thiol moieties, or four moieties, or more than four moieties. In embodiments, the crosslinker has a molecular weight of between 100 to 800 g/mol, for example, between 200 to 700 g/mol, or 300 to 600 g/mol, or 400 to 500 g/mol. The crosslinker may be pentaerythritol tetrakis(3-mercaptopropionate), comprising the formula (vii):

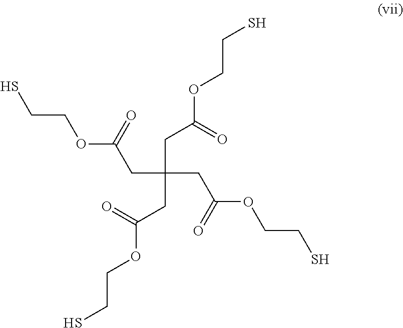 [SMILES: O=C(CC(CC(=O)OCCS)(CC(=O)OCCS)CC(=O)OCCS)OCCS]

Alternatively, the reactive diluent may comprise a different moiety that is capable of reacting with the moiety on the crosslinker to produce a covalent bond between the crosslinker and the reactive diluent, and/or between the crosslinker and the prepolymer. For example, the crosslinker may comprise an unsaturated side-chain, for example, an alkyne, and the reactive diluent may comprise an azide group. Alternatively, the crosslinker may comprise an alkene moiety and the reactive diluent may comprise a thiol moiety.

In embodiments, the reactive diluent may be present in a quantity of between 0 and 50 w/w % of the total composition, for example, between 5 and 45 w/w %, or 10 and 40 w/w %, or 15 and 35 w/w %, or 20 and 30 w/w % or 25 w/w %. For example, the resin composition may comprise the reactive diluent in a quantity of 20, 21, 22, 23, 24, 25, 26, 27, 28, 29, 30, 31, 32, 33, 34, 35, 36, 37, 38, 39, or 40 w/w %.

In embodiments, the prepolymer may be present in a quantity of between 10 and less than 100 w/w % of the total composition, for example, between 20 and 90 w/w %, or 40 and 80 w/w %, or 60 and 70 w/w %. For example, the resin composition may comprise the prepolymer in a quantity of 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 w/w %. In embodiments, the resin composition comprises the prepolymer is present in the resin composition in a quantity of 60 w/w %.

In embodiments, the cross-linker may be present in a quantity of between 0 and 50 w/w % of the total composition, for example, between 5 and 45 w/w %, or 10 and 40 w/w %, or 15 and 35 w/w %, or 20 and 30 w/w % or 25 w/w %. For example, the resin composition may comprise a total quantity of cross-linker of 20, 21, 22, 23, 24, 25, 26, 27, 28, 29, 30, 31, 32, 33, 34, 35, 36, 37, 38, 39, or 40 w/w % In an embodiment, the resin composition comprises 1 mass equivalent of prepolymer (M_{P}), 1 mass equivalent of reactive diluent (M_{D}), 1 mass equivalent of crosslinker (M_{D}), and optionally up to 5, 10, 15, 20, 25 or 30 w/w % viscosity modifier of the total composition, and 1 w/w % photoinitiator of the total composition.

Alternatively other mass equivalents can be used, for example from 0.25M_{P}:5.0M_{D} to 5.0M_{P}:0.25M_{D}, and/or 0.25M_{P}:5.0M_{C} to 5.0M_{P}:0.25M_{C} and/or 0.25M_{D}:5.0M_{C} to 0.25M_{D}:5.0M_{C}.

In embodiments, the resin composition may comprise a ratio of allyl groups to thiol groups, wherein the ratio is between 1 to 2 molar equivalents of allyl groups to 1 molar equivalent of thiol groups. For example, the resin composition may comprise a ratio of 2.0, 1.5, or 1.0 molar equivalents of allyl groups to 1.0 molar equivalent of thiol groups. In embodiments, the resin composition may comprise up to 30, 25 or 20 w/w % viscosity modifier of the total composition. In embodiments, the resin composition comprises 1 w/w % photoinitiator of the total composition.

A yet further aspect of the invention provides a method of fabricating a resin composition, the method comprising combining a prepolymer, a reactive diluent, and optionally a crosslinker, wherein the reactive diluent has an absorption maximum (λₘₐₓ) between 350 and 520 nm in a UV/Vis spectrum in a solvent (e.g. acetone).

A yet further aspect of the invention provides a resin composition that is used to fabricate a cross-linked polymer, for example a shape memory polymer, i.e. the cross-linked polymer may comprise a permanent state and a temporary state, the permanent state being capable of undergoing a morphological change to the temporary state, or vice versa, upon induction by an external stimulus. The external stimulus may be a temperature change, for example, a temperature change approximately at a physiological temperature. The external stimulus may comprise one or more of direct or Joule heating, radiation and laser heating, microwaves, pressure, moisture, the presence or absence of solvent or solvent vapours, and/or change in pH. The resin composition may be as set out above.

The cross-linked polymer of the invention may be degradable, i.e. the polymer may degrade into degradation products that are metabolised or excreted under physiological conditions without causing harm. The cross-linked polymer may exhibit degradation via surface erosion. The cross-linked polymer may degrade upon exposure to, for example, water, heat, a change in pH (e.g. from exposure to acid or base), or another chemical change or physical force. The cross-linked polymer may degrade into non-toxic by-products, for example, non-toxic small molecule by-products, for example, oligomers and/or monomers e.g. carbonate monomers, carbonate urethanes, diols, carbamates, and/or urethanes.

Advantageously, controlling the amount or number of carbonate linkages in the composition enables the degradability of the resulting cross-linked polymer to be controlled. Also, controlling the amount or number of urethane and/or urea linkages in the composition enables the shape memory behaviour of the cross-linked polymer to be controlled.

A yet further aspect of the invention provides a device that is fabricated from the cross-linked polymer. The device may be fabricated using an additive manufacturing technique or apparatus. For example, the device may be fabricated using stereolithography, or microstereolithography.

The device may be a 4D printed device, i.e. the device is fabricated using an additive manufacturing technique such as 3D printing to produce a primary shape, and the device is further deformed and/or manipulated to produce a secondary shape. The secondary shape may a flexible and/or deployable shape, for example, a minimally invasive shape for minimally invasive delivery to a site within a patient. The device may be manufactured using machining techniques, for example, turning, milling, and/or drilling techniques. The device may comprise or be a complex product that may be assembled by hand, through assembling simpler parts into a complex product, and/or fixation and/or adhering.

The device may be for a biomedical application, and/or may be a medical device, and/or an implantable medical device. For example, the device may be used in cardiovascular, orthopaedic, surgical, or rehabilitative applications. The device may be a vascular device, and/or a device for cardiac defects. The device may be an absorbable plate, a screw, an interbody spacer and/or another resorbable device. The device may be a stent, e.g. a stent for coronary, peripheral, nasal and auditory applications and so on. The stent may possess shape memory properties, i.e. conforming to local tissue. The device may be used for systemic and/or local drug delivery, e.g. transdermal drug delivery, e.g. in postoperative pain management, or in anti-infective absorbable implants. The device may be used in tissue engineering, in microneedles and/or in vaccine deliveries. The device may be an eluting device, e.g. an implant and/or a drug delivery device.

The device may be a tissue scaffold, for example, a porous tissue scaffold. The device may be porous. In embodiments, the pore sizes may range from approximately 200 μm to 1500 μm.

Alternatively, the device may be used in self-fitting electronics, for example, 3D printed electronic sensors, and/or biosensors.

A yet further aspect of the invention provides a method of fabricating a cross-linked polymer, the cross-linked polymer comprising at least one unsaturated side-chain, the method comprising:
i. providing a resin composition of the invention as set out above;
ii. cross-linking the prepolymer.

Preferably, step (ii) cross-linking the pre-polymer is performed by contacting the resin composition with an initiator. Preferably, an energy source is provided to activate the initiator.

The method may comprise contacting the resin composition with a catalyst and/or an initiator. For example, the catalyst and/or initiator may be a photoinitiator. The method may comprise exposing the resin composition comprising a photoinitiator to an energy source, for example, a light source, for example, UV light.

The initiator may be a photoinitiator, e.g. a bis acyl phosphine. Suitable photoinitiators include those sold under the trade name Irgacure® by BASF, for example, Irgacure 819, or those sold under the trade name Omnicat® photoinitiators by IGM resins.

The initiator may be a radical initiator, for example, a peroxide such as hydrogen peroxide, or an organic peroxide such as benzoyl peroxide. The radical initiator may be an azo compound, for example, AlBN or ABCN. In embodiments, the energy source may be heat, i.e. the reaction may be initiated thermally.

The initiator may be present in a quantity of between 0 and 5 w/w % of the total composition, for example, up to 4 w/w %, or up to 3 w/w %, or up to 2 w/w %, or up to 1 w/w % of the total composition, for example, 0.5 w/w % of the total composition. The initiator, e.g. the photoinitiator, may be present in a quantity of 0.5, 1.0, 1.5, 2.0, 2.5, 3.0, 3.5, 4.0, 4.5, or 5.0 w/w % of the total composition.

The method may be performed in or by an apparatus for 3D printing, e.g. an apparatus for stereolithography.

The cross-linked polymer may be further functionalised. The further functionalisation may take place post polymerisation, i.e. after the cross-linked polymer has been fabricated from the resin composition. The cross-linked polymer may comprise unsaturated side-chains after the cross-linking process has taken place. The method may comprise further functionalisation of these unsaturated side chains. For example, the method may comprise cross-linking a polymer in an additive manufacturing process, e.g. a 3D printing process and/or a stereolithography process, and further providing reagents to functionalise the cross-linked polymer, e.g. the surface of the cross-linked polymer. The functionalisation of the cross-linked polymer may take place in a separate step.

In embodiments, the method may further comprise step iii. providing a reagent for halogenation of at least one unsaturated side chain of the cross-linked polymer. The reagent may be a diatomic halogen, e.g. chlorine, bromine and/or iodine, or a halogenating reagent, e.g. a hypohalous acid such as HOCl, HOBr, HOI, or a Brønsted acid, e.g. HF, HCl, HBr, and/or HI.

Additionally or alternatively, the method may further comprise step iv. providing a reagent for alkylation of the at least one unsaturated side chain. The reagent may be an alkylating agent, e.g. an alkyl halide, or an alkyl thiol.

Additionally or alternatively, the method may further comprise step v. providing a reagent for functionalising the at least one unsaturated side chain with a hydrophobic moiety. The hydrophobic moiety may increase the hydrophobicity of the cross-linked polymer. The hydrophobic moiety may comprise an alkyl chain, for example, a linear alkyl chain comprising between 8 and 15 carbons, say 10 carbons, or 9, 10, 11, 12, 13, 14, or 15 carbons. In embodiments, the reagent may be a compound comprising a thiol moiety, e.g. an alkyl or aryl thiol compound, that is capable of adding across an unsaturated side-chain, e.g. an alkene moiety.

Additionally or alternatively, the method may further comprise a step for providing a reagent for functionalising the at least one unsaturated side chain with a hydrophilic moiety. The hydrophilic moiety may increase the hydrophilicity of the cross-linked polymer. The hydrophilic moiety may comprise one or more carboxylic acid groups, and/or one or more hydroxyl groups. The hydrophilic moiety may comprise an alkyl chain comprising one or more carboxylic acid groups and/or one or more hydroxyl groups. In embodiments, the reagent may be a compound comprising a thiol moiety comprising hydrophilic groups, e.g. an alkyl or aryl thiol compound comprising hydrophilic side groups, that is capable of adding across, and/or reacting with, an unsaturated side-chain, e.g. an alkene moiety to form a covalent bond.

Alternatively, the unsaturated side-chains of the cross-linked polymer may be further functionalised in other types of reaction. For example, the one or more unsaturated side-chain of the cross-linked polymer may be an alkene, and may react in a cycloaddition, e.g.

a Diels-Alder reaction. Other atoms or moieties may be added across or to the unsaturated side chains. For example, the unsaturated side-chain may be an alkene that undergoes an epoxidation or a cyclopropanation.

Additionally or alternatively, the method may further comprise a step for providing a reagent for functionalising the at least one unsaturated side chain with a tag, for example, a fluorescent tag, a radioactive tag, or a biomolecule tag, for labelling or detection of the cross-linked polymer. This is particularly useful if the cross-linked polymer is fabricated into a medical device for implantation into a patient.

Additionally or alternatively, the method may further comprise step vi. providing a reagent for functionalising the at least one unsaturated side chain with a biomolecule, for example, a protein, and/or a cell adhesion moiety, e.g. a cell adhesion molecule (CAM). The biomolecule may be involved in adhesion or binding to physiological targets. For example, a cell adhesion molecule (CAM) may be involved in binding to cells, e.g. bone cells within a tissue scaffold, or to the extracellular matrix. For example, the further functionalised cross-linked polymer may comprise a functionalised surface to elicit a specific cellular response.

The steps iii, iv, v, and/or vi of the method may be performed at the same time as the resin composition is fabricated into a cross-linked polymer, e.g. during additive manufacture, or may be performed after the resin composition has been fabricated into a cross-linked polymer in a separate step, i.e. after steps i to ii of the method. Only one of the steps iii, iv, v, and/or vi may be performed after steps i to ii have been performed. Alternatively, two or more of the steps may be selected to be performed, either consecutively or concurrently, after steps i to iii have been performed. For example, the method may comprise steps i to ii, followed by step iii and further followed by step vi.

Additionally or alternatively, the monomers of the prepolymer may undergo further functionalisation. The monomers of the prepolymer may be functionalised before polymerisation into the prepolymer. The monomers of the prepolymer may be functionalised after polymerisation into the prepolymer, but before cross-linking into a cross-linked polymer.

Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible. For the avoidance of doubt, the terms “may”, “and/or”, “e.g.”, “for example” and any similar term as used herein should be interpreted as non-limiting such that any feature so-described need not be present. Indeed, any combination of optional features is expressly envisaged without departing from the scope of the invention, whether or not these are expressly claimed. The applicant reserves the right to change any originally filed claim or file any new claim accordingly, including the right to amend any originally filed claim to depend from and/or incorporate any feature of any other claim although not originally claimed in that manner.

Embodiments of the invention will now be described by way of example only with reference to the accompanying drawings in which:

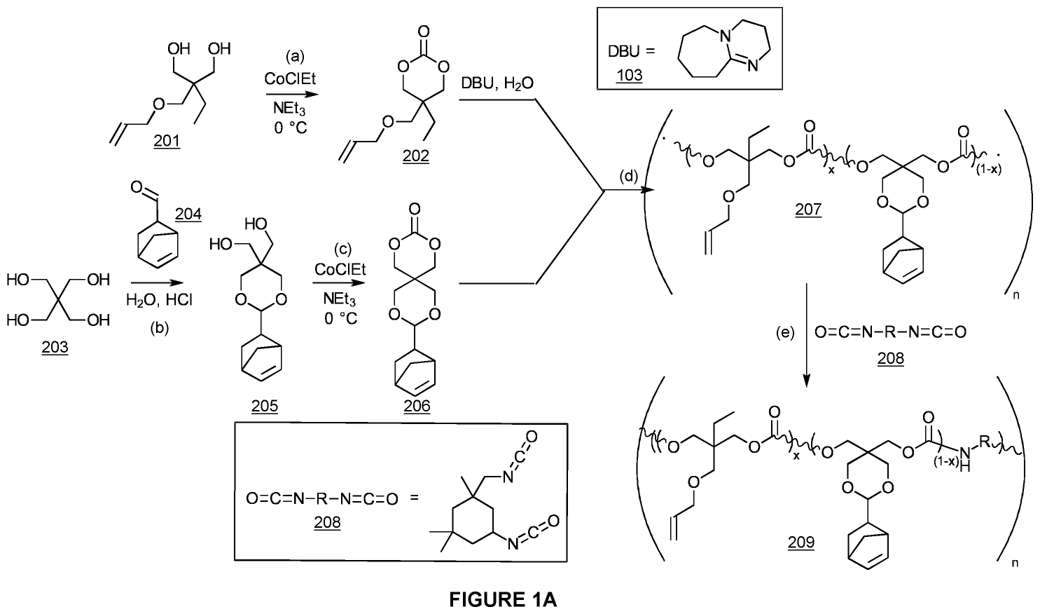

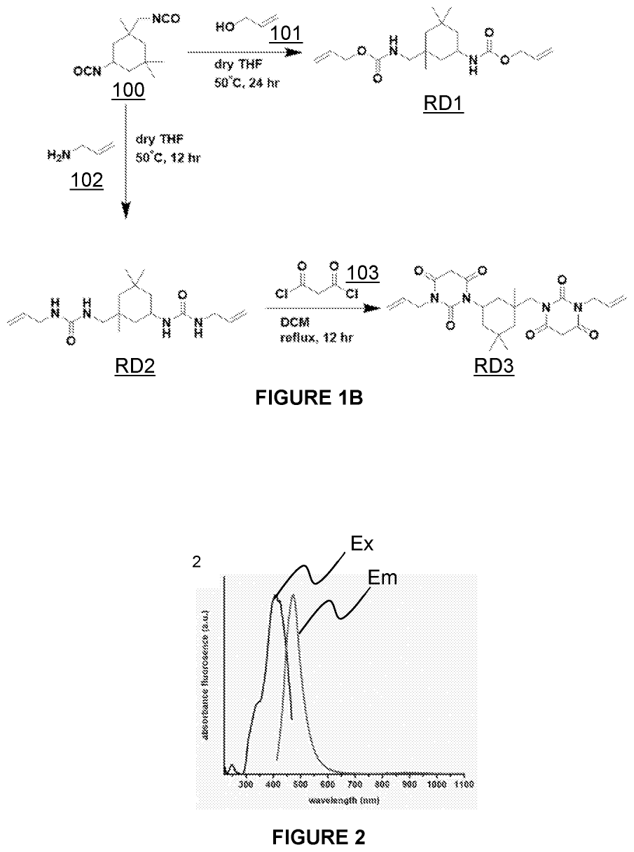

FIG. 1A is a synthetic pathway to a prepolymer for use in the resin composition of the invention;

FIG. 1B is synthetic pathway to Reactive Diluents 1 and 2 for use in resin compositions of Comparative Examples of the invention, and Reactive Diluent 3 for use in resin compositions of Examples of the invention;

FIG. 2 is a UV-vis absorption spectrum and a fluorescence emission spectrum for Reactive Diluent 3;

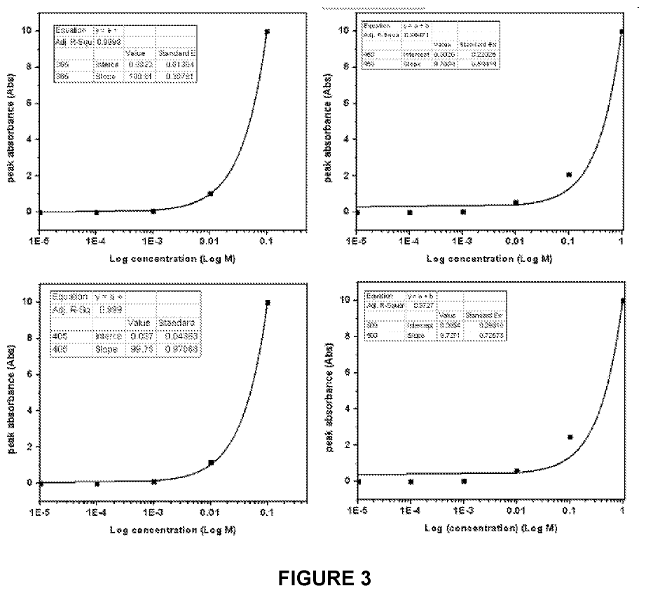

FIG. 3 is a series of graphs illustrating the absorption behaviour of the resin components;

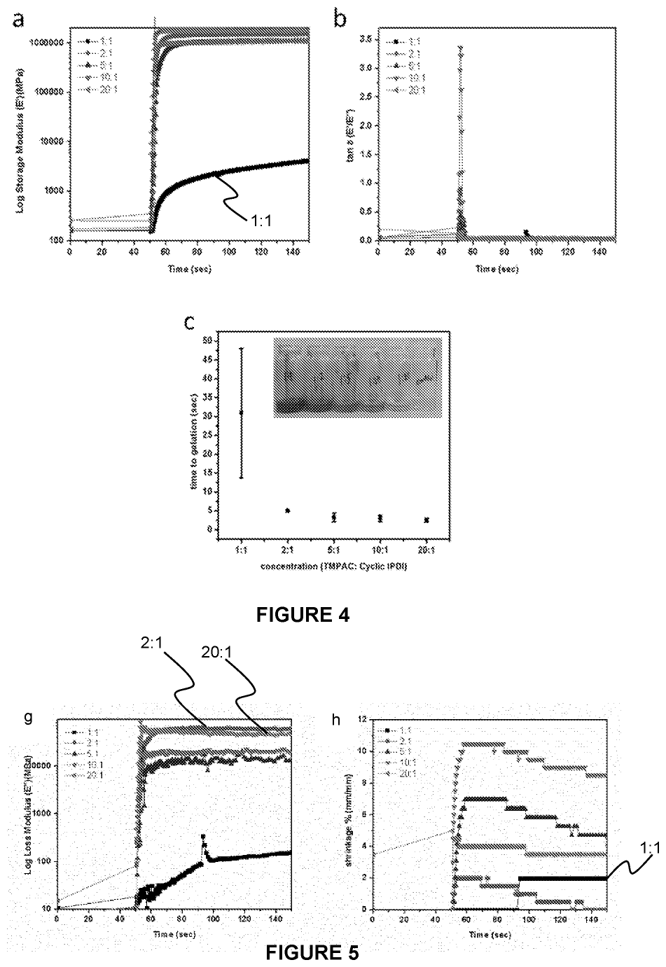

FIG. 4 is a series of graphs showing the results of photorheology experiments to illustrate the gelation kinetics of resin compositions containing Reactive Diluent 3 at different concentrations and 1 wt. % photoinitiator;

FIG. 5 is a series of graphs showing the loss modulus and the normalized shrinkage of resin compositions containing Reactive Diluent 3 at different concentrations;

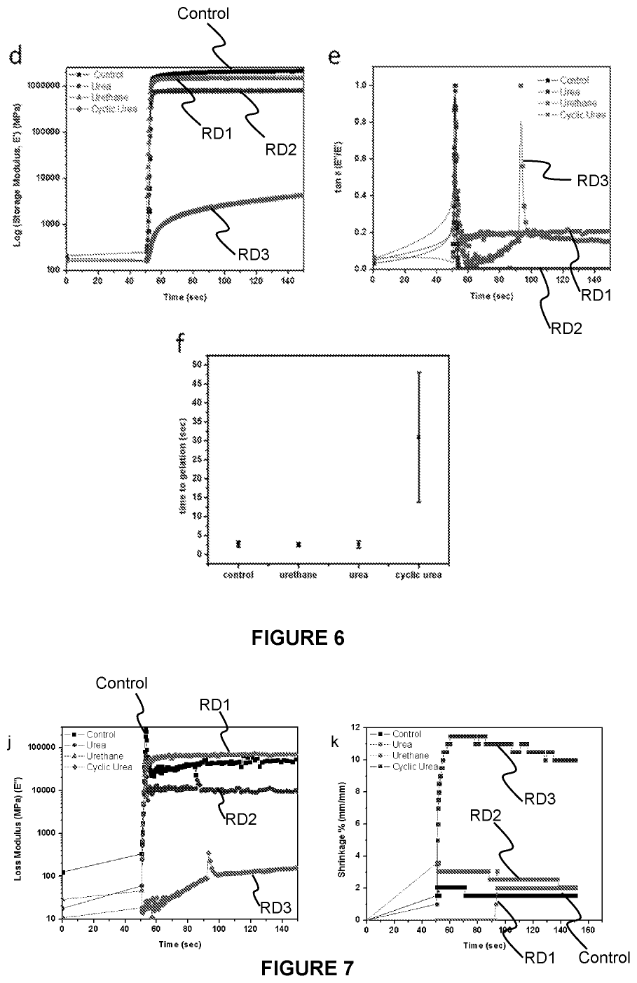

FIG. 6 is a series of graphs showing the results of photorheology experiments to illustrate the gelation kinetics of resin compositions containing Reactive Diluents 1, 2, and 3 and 1 wt. % photoinitiator;

FIG. 7 is a series of graphs showing the loss modulus and the normalized shrinkage of resin compositions containing Reactive Diluent 1, 2, or 3;

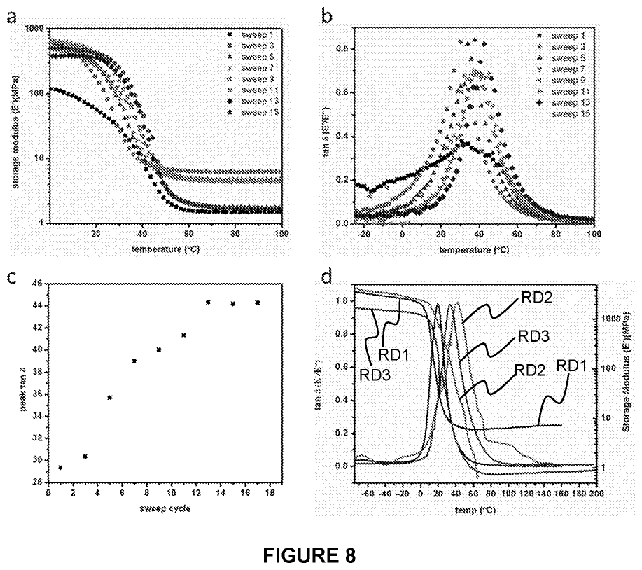

FIG. 8 is a series thermal sweeps examining storage moduli and loss factors;

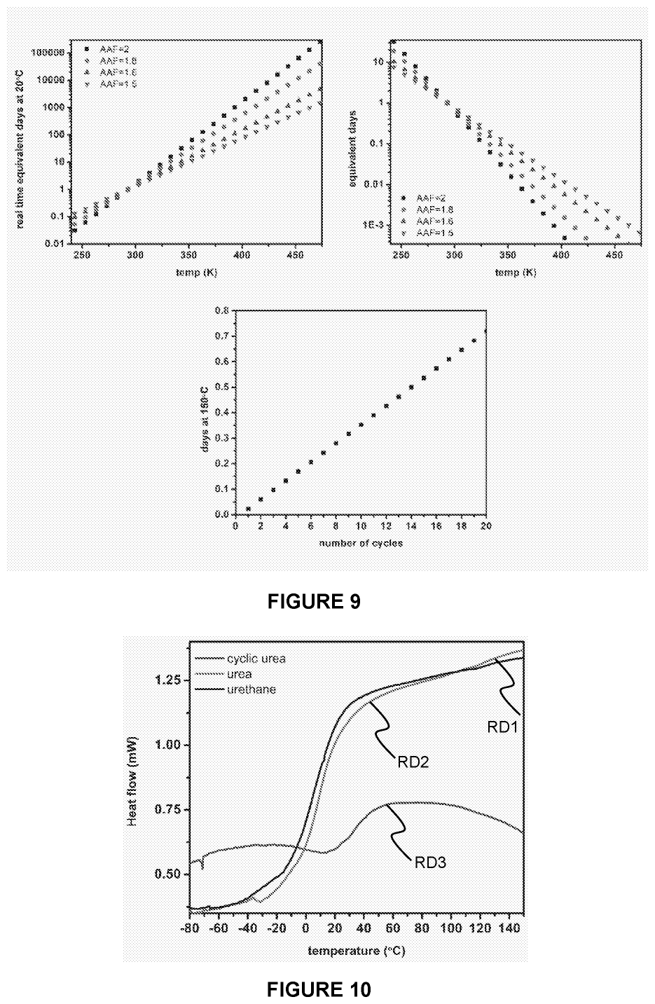

FIG. 9 is shown Arrhenius approximations for comparison with the results shown in FIG. 8;

FIG. 10 is representative thermograms measured by DSC (Differential Scanning calorimetry) for 2 mg samples of the resin compositions containing one of Reactive Diluent 1, 2, or 3;

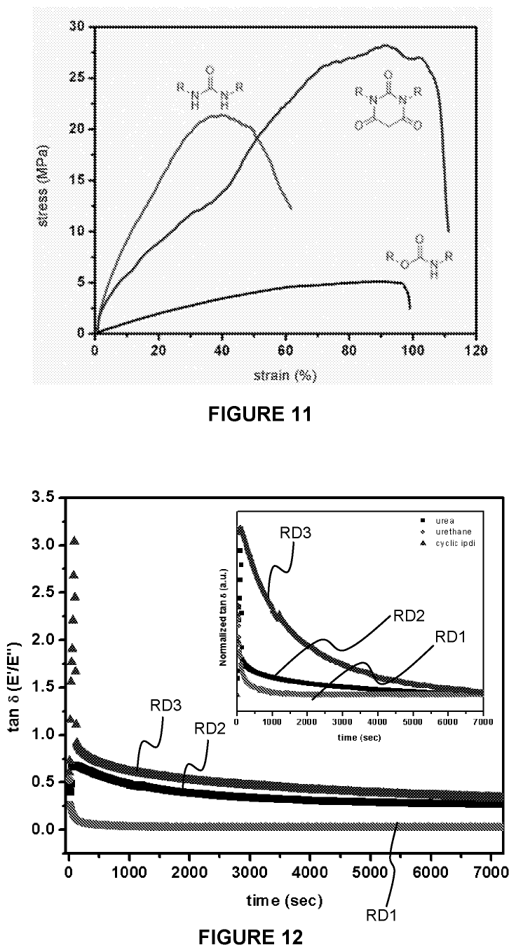

FIG. 11 is a graph showing representative stress strain behaviour when examined using tensile loading of cured resin compositions containing the Reactive Diluents 1, 2, and 3 respectively in a 1:1 ratio with Prepolymer 1;

FIG. 12 is a graph showing the relaxation kinetics displaying the change in storage modulus (E′) and tan δ of printed scaffolds immersed in PBS at 37° C.;

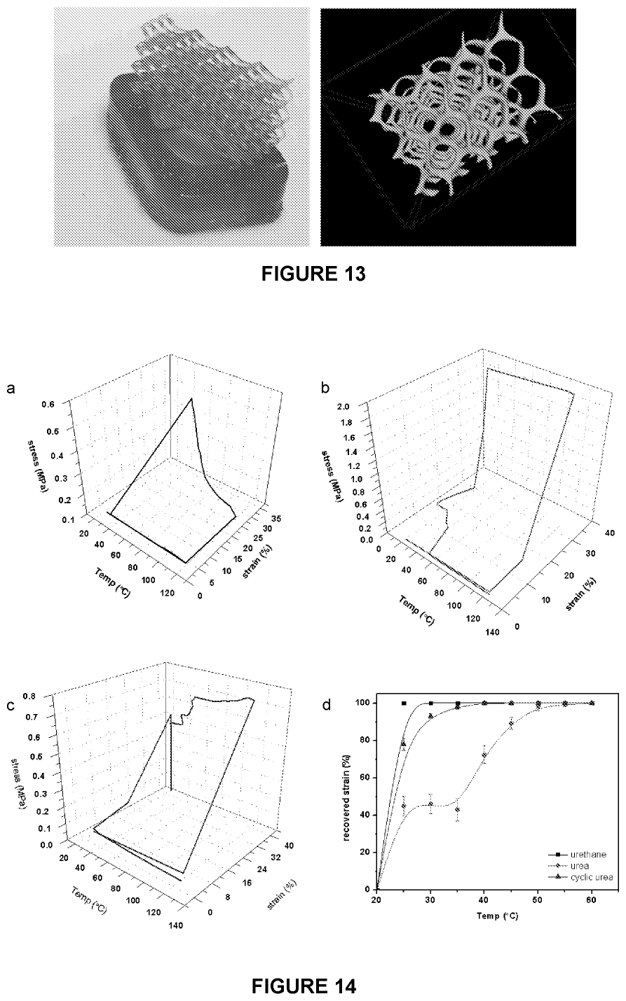

FIG. 13 is a representative porous tissue scaffold printed by stereolithography;

FIG. 14 is a series of graphs showing the shape memory behaviour of printed porous scaffolds formed from resin compositions comprising Reactive Diluents 1, 2, and 3

Referring first to FIG. 1A, there is shown there is shown a synthetic route to a prepolymer (209) for use in a resin composition, according to an embodiment of the invention. In this embodiment, the prepolymer (209) was fabricated in chain extension reaction (e) from a polycarbonate oligomer (207) and a diisocyanate (208) to produce the prepolymer (209), which is a mixed polycarbonate polyurethane oligomer. In this embodiment, the diisocyanate (208) is isophorone diisocyanate (IPDI) (208). The prepolymer (209) had molecular weights of less than or equal to 3 kDa and polydispersity indices (PDI) of 1.4.

The polycarbonate (207) was synthesised in ring opening polymerisation reaction (d) from first cyclic carbonate (202) and second cyclic carbonate (206) in the presence of water and a DBU initiator (103), as described in WO2018/229456A1. The reaction (d) of first cyclic carbonate (202) and second cyclic carbonate (206) yielded oligomers of polycarbonate (207) with lengths of below 1.2 kDa with PDIs of below 1.2.

In this embodiment, first cyclic carbonate (202) is 5-[(allyloxy)methyl]-5-ethyl-1,3-dioxan-2-one, and second cyclic carbonate (206) is 9-(5-norbornen-2-yl)-2,4,8,10-tetraoxa-3-spiro[5.5]undecanone, which were synthesised in accordance with the protocols described in IA Barker et. al., Biomaterials Science, 2014, 2, 472-475; and also in Y He et. al., Reactive and Functional Polymers, Vol. 71, Issue 2, February 2011, p. 175-186.

First cyclic carbonate (202) was synthesised in one step, in reaction (a) from diol (201) and propionyl chloride in the presence of triethylamine at 0° C. In this embodiment, diol (201) is 2-[(allyloxy)methyl]-2-ethyl-1,3-propanediol.

Second cyclic carbonate (206) was synthesised in two steps, using polyol (203) as the starting material. In reaction (b), polyol (203) and aldehyde (204) underwent reaction in the presence of hydrochloric acid to produce diol (205). Diol (205) underwent subsequent reaction, in reaction (c), with propionyl chloride in the presence of triethylamine at 0° C. to produce the second carbonate (206). In this embodiment, polyol (203) is pentaerythritol, aldehyde (204) is bicyclo[2.2.1]hept-5-ene-2-carboxaldehyde, and diol (205) is [5-(hydroxymethyl)-2-(5-norbornen-2-yl)-1,3-dioxan-5-yl]methanol.

The prepolymer for use in the resin composition according to the invention may be one or more of prepolymer (209) (which contains an O═C—N linkage), polycarbonate (207) (a copolymer of two different carbonate monomers, (202) and (206)), or a homopolymer synthesised from either first cyclic carbonate (202) or second cyclic carbonate (206).

Referring now to FIG. 1B, there is shown a synthetic pathway to Reactive Diluent 1, 2, and 3. Reactive Diluents 1 and 2 do not exhibit an absorption maximum (λₘₐₓ) of between 350 and 520 nm in acetone, and as such, were used in resin compositions according to Comparative Examples of the invention. Reactive diluents of the invention are non-white coloured. For example, Reactive Diluent 3 is orange in colour and exhibits an absorption maximum (λₘₐₓ) of ca. 450 nm in a UV/Vis spectrum in acetone, and as such, was used in resin composition according to Examples of the invention.

All three reactive diluents were synthesised from a common starting material; isophorone diisocyanate (100).

Reactive Diluent 1 (RD1) was produced by reaction of isophorone diisocyanate (100) with allyl alcohol (102) in dry THF (tetrahydrofuran) at 50° C. for 24 hours. Reactive Diluent 2 (RD2) was produced by reaction of isophorone diisocyanate (100) with allylamine (102) in dry THF at 50° C. for 12 hours. Reactive Diluent 3 (RD3) was produced by reaction of Reactive Diluent 2 (RD2) with malonyl chloride (103) in DCM (dichloromethane) at reflux for 12 hours.

Referring now to FIG. 2, there is shown a UV-vis absorbance spectra (Ex) and a fluorescence emission spectra (Em) for Reactive Diluent 3 (2b).

The spectra 2a and 2b of Reactive Diluent 3 were recorded in spectroscopy grade acetone, diluted from 1M stock solutions down to 10 μM using serial dilutions. Absorption behaviour was examined from 200 nm to 800 nm, at 1 nm increments, using a ThermoFisher 350 UV-vis spectrophotometer (ThermoFisher Scientific, UK). Absorption maxima at 365, 405, 455, and 500 nm were recorded for comparison.

Referring also to FIG. 3, there is shown a series of graphs illustrating the absorption behaviour of the resin components in spectroscopy grade acetone, diluted from 1 M stock solutions down to 10 μM using serial dilutions. Absorption behaviour was examined from 200 nm to 800 nm, at 1 nm increments, with absorption maxima quantified at 365, 405, 455, and 500 nm, as indicated in the box in each graph.

This was used to determine the concentration at which Reactive Diluent 3 would act as a photoinhibitor for wavelengths of interest. At 365 nm, the inflection point of the logarithmic plot of the linear fit was located at ca 0.015M; 405 nm inflection point was located at 0.0131M; 450 nm inflection point was found at 0.099M; 500 nm inflection point was located at 0.1312M. These results indicate that for incorporating Reactive Diluent 3 into a resin composition to function as a photoinhibitor, at least 0.015 M is needed.

The invention is exemplified by the following non-limiting Examples.

Synthesis of Reactive Diluents 1 to 3

All chemicals were commercially available and used without further purification unless otherwise stated. Solvents were of ACS grade or higher. NMR spectra (400 MHz for ¹H and 125 MHz for ¹³C) were recorded on a Bruker 400 spectrometer, with MestReNova v9.0.1 (Mestrelab Research, S.L., Santiago de Compostela, Spain) used to process spectra. Chemical shifts were referenced to CDCl₃ at 7.26 ppm (¹H) and 77.16 ppm (¹³C), and DMSOd₆ at 2.50 ppm (¹H) and 39.51 ppm (¹³C).

Synthesis of Reactive Diluent 1 (Comparative Example): Isophorone diisocyanate (55.53 g, 0.250 moles) was added by canula transfer to a round bottomed flask (dried 120° C. overnight and sealed) under negative pressure, followed by dry THF. Freshly distilled allyl alcohol (30.64 g, 0.53 moles), stored over molecular sieves, was transferred dropwise into the flask while stirring at 300 rpm. Upon complete transfer of the allyl alcohol, the reaction was heated to 50° C. and held isothermally for 24 h, at which point residual diisocyanate was quenched with water (at 50° C.). The crude product was obtained after dissolving the reaction mixture in ethyl acetate, washing with 1M HCl (3 washes) and brine (once) and concentrating the product. A viscous clear oil was collected after column chromatography (90:10 EtoAc:Hexane) and concentrated in vacuo. ¹H NMR (CDCl₃): 0.83-0.92 (4), 0.99-1.05 (4), 1.17-1.21 (2), 1.36-1.40 (2), 1.67-1.74 (3), 1.85-1.88 (2), 2.91-2.92 (2), 3.22, 3.28-3.33 (4), 3.79-3.81 (2), 4.14-4.15 (2), 4.53-4.55 (2), 4.84, 5.18-5.31 (4), 5.85-5.94 (m). ¹³C NMR (CDCl₃): 23.3, 27.7, 29.8, 32.0, 35.1, 36.5, 412.0, 44.8, 46.4, 47.2, 55.0, 65.7, 117.8, 133.0, 155.6, 156.8 ppm.

Synthesis of Reactive Diluent 2 (Comparative Example): Isophorone diisocyanate (55.53 g, 0.250 moles) was added to a round bottomed flask (dried 120° C. overnight) and THF. The flask was chilled to 0° C. for 1 h and held isothermally for the slow dropwise addition of allylamine (30.24 g, 0.53 moles), followed by 1 h of stirring before the mixture was allowed to heat to ambient temperature for 12 h, after which the solution was heated to 50° C. for 24 h. The solid product was dissolved in ethyl acetate, washed with 1M HCl (3 washes) and brine (1 wash), and the organic solvent removed to produce off-white crystals. White crystals of the product were obtained through recrystallization, in isopropyl alcohol and THF. ¹H NMR (DMSOd₆): 0.77-0.82, 0.85-0.99 (m), 1.07-1.11, 1.34-1.37 (2), 1.44-1.53 (4), 2.70-2.81 (m), 3.37, 3.61-3.70, 4.99-5.01, 5.06-5.12, 5.70-5.80, 5.88, 6.0-6.1 (NH); ¹³C NMR (DMSOd₆): 15.3, 23.3, 23.6, 27.9, 31.9, 36.4, 46.3, 46.7, 56.8, 65.8, 101.3, 115.3, 158.4, 159.4 ppm.

Synthesis of Reactive Diluent 3 (Example of the Invention): A slight excess of malonyl chloride (12.21 g, 0.087 moles) was added dropwise into a solution of Reactive Diluent 2 (12.00 g, 0.04 moles) in dichloromethane (300 mL) under ambient conditions with stirring. The solution became brownish with the addition, and was heated to reflux (70° C.) overnight.

The solution turned red during the reaction and, after cooling, was washed with 1 M HCl_{aq} (3×), and once with water before being concentrated in vacuo to yield a dark red oil. ¹H NMR (DMSOd₆): 0.64-0.68, 0.75-0.80, 0.86-0.97, 1.14-1.18, 1.28-1.46, 1.59-1.65, 1.78, 1.86-2.11, 2.36-2.38, 3.12, 3.25, 3.44-3.54, 3.60-3.76, 3.94-3.99, 4.13-4.29, 4.74-4.94, 4.92-5.10, 5.59-5.74; ¹³C NMR (DMSOd₆).

Synthesis of Prepolymer 1

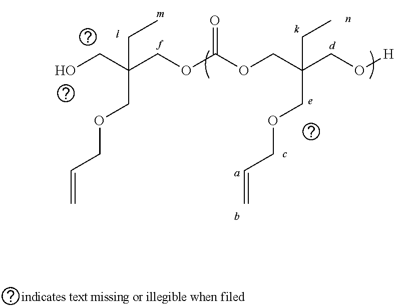

Prepolymer 1 was synthesised by ring opening polymerisation using a DBU catalyst from 5-[(allyloxy)methyl]-5-ethyl-1,3-dioxan-2-one according to the method described in IA Barker et. al., Biomaterials Science, 2014, 2, 472-475; and also in Y He et. al., Reactive and Functional Polymers, Vol. 71, Issue 2, February 2011, p. 175-186 to obtain approximately 2 kDa PolyTMPAC oligomers.

Resin Composition According to Examples and Comparative Examples of the Invention

General Procedure: Prepolymer, Reactive Diluent, and Crosslinker (pentaerythritol tetrakis(3-mercaptopropionate)) were added to a vial in stoichiometric amounts. Propylene carbonate (20 wt. % of the total resin composition) was used to reduce resin viscosity and aid in mixing. After homogenization, the resin was placed in a brown glass container and stored at room temperature. Photoinitiator (1 wt. % of the total resin composition) was added. Paprika Extract was added to resin compositions containing Reactive Diluent 1 or Reactive Diluent 2.

Resin Composition according to Example of Invention: Reactive Diluent 3 (13.78 g), Prepolymer 1 (15.28 g), 1,3,5-triallyl-1,3,5-triazine-2,4,6(1H,3H,5H)-trione (14.65 g), Crosslinker (pentaerythritol tetrakis(3-mercaptopropionate)) (24.41 g), and propylene carbonate (16.54 g) were mixed together. Irgacure 819 (0.82 g) was added to the resulting mixture. Table 1 shows resin compositions according to Examples and Comparative Examples of the invention.

TABLE 1
Resin Compositions according to Examples and Comparative
Examples of the Invention (amounts in grams)

| | | Reactive | Reactive | Reactive Diluent 3 | | | | |
|---|---|---|---|---|---|---|---|---|
| | Control | Diluent 1 | Diluent 2 | 1:01 | 1:02 | 1:05 | 1:10 | 1:100 |
| Prepolymer 1 | 0.2000 | 0.2000 | 0.2000 | 0.2000 | 0.2000 | 0.2000 | 0.2000 | 0.2000 |
| Reactive | 0.0000 | 0.2000 | 0.2000 | 0.2000 | 0.1000 | 0.0400 | 0.0200 | 0.0020 |
| Diluent | | | | | | | | |
| 1,3,5-triallyl- | 0.2000 | 0.2000 | 0.2000 | 0.2000 | 0.2000 | 0.2000 | 0.2000 | 0.2000 |
| 1,3,5-triazine- | | | | | | | | |
| 2,4,6(1H,3H,5H)- | | | | | | | | |
| trione | | | | | | | | |
| Crosslinker | 0.3989 | 0.5373 | 0.5382 | 0.4980 | 0.4485 | 0.4188 | 0.4088 | 0.3998 |
| Mass of | 0.7989 | 1.1373 | 1.1382 | 1.0980 | 0.9485 | 0.8588 | 0.8288 | 0.8018 |
| Reactive | | | | | | | | |
| Components | | | | | | | | |
| Irgacure 819 | 0.0080 | 0.0114 | 0.0114 | 0.0110 | 0.0095 | 0.0086 | 0.0083 | 0.0080 |
| Inhibitor | 0.0080 | 0.0114 | 0.0114 | NA | NA | NA | 0.0020 g | 0.0040 |

Analysis of Resin Compositions and Crosslinked Polymers According to the Invention

Referring now to FIG. 4, there is shown a series of graphs (a to c) showing the results of photorheology experiments to illustrate the gelation kinetics of resin compositions containing Reactive Diluent 3 and 1 wt. % photoinitiator (Irgacure® 819).

The resin compositions were exposed to 350 nm to 520 nm light at ambient temperature and humidity, using parallel plate oscillatory shear tests by shearing between two parallel plates, one made of glass and transparent (¹ Hz shear, 1% oscillation), where a steady state behaviour was established over a 50 s period without irradiation, followed by irradiation at 350 nm to 520 nm at 10 mW·cm⁻² at ambient conditions (20° C.). Measurements were taken every 0.2 s over the course of 120 s. The inflection points of the moduli plots, and the peak tan δ values, were used to determine the time to gelation of the resin. Sample shrinkage was measured by measuring the distance between the plates at the same sampling rate as the other metrics.

Graph ‘a’ of FIG. 4 shows the storage modulus for concentration ratios ranging from 1:1 to 1:100 Reactive Diluent 3: Prepolymer 1. Graph ‘b’ of FIG. 4 shows the peak tan δ values as a function of Reactive Diluent 3 concentration in the resin composition. Graph c of FIG. 4 shows the time to gelation as a function of the concentration of Reactive Diluent 3 in the resin composition. The most rapid gelation occurred with the lowest concentration of Reactive Diluent 3.

It is shown that increasing the concentration of the Reactive Diluent 3 resulted in the final E′ amplitude decreasing while the time to the final E′ and the maximum loss factor value increased. Only 1:1 ratio of Reactive Diluent 3 with Prepolymer 1 were found to result in substantial inhibition (as determined by a change in the time to a phase change in the material or the plateaued storage modulus); lower concentrations did not affect the time to final storage moduli (as shown in Graph a) nor the time to peak phase transition (as shown in Graph b and c).

Referring also to FIG. 5, there is shown graph ‘g’ and graph ‘h’. Graph ‘g’ shows the loss modulus for concentration ratios ranging from 1:1 to 1:20 Reactive Diluent 3: Prepolymer 1. Graph ‘h’ shows the normalized shrinkage of the gelled resin composition containing different ratios of Reactive Diluent 3 to Prepolymer 1. No trend was found.

Referring now to FIG. 6, there is shown a series of graphs (d to f) showing the results of photorheology experiments to illustrate the gelation kinetics of resin compositions containing Reactive Diluents 1, 2, and 3 and 1 wt. % photoinitiator (Irgacure® 819) (RD1, RD2, RD3 respectfully).

Resin samples crosslinking kinetics were examined as a function of gelation time by measuring the dampening or phase ratio (tan δ), storage moduli, loss moduli, complex viscosity, and film thickness during photorheology. To conduct these experiments, we used an Anton Paar rheometer (Anton Paar USA Inc, Ashland, VA, USA) fitted with a detachable photoillumination system with two parallel plates (10 mm disposable aluminum hollow shaft plate, Anton Paar).

The resin compositions were exposed to 350 nm to 520 nm light at ambient temperature and humidity, using parallel plate oscillatory shear tests by shearing between two parallel plates, one made of glass and transparent (¹ Hz shear, 1% oscillation), where a steady state behaviour was established over a 50 s period without irradiation, followed by irradiation at 350 nm to 520 nm at 10 mW·cm⁻² at ambient conditions (20° C.). Measurements were taken every 0.2 s over the course of 120 s. The inflection points of the moduli plots, and the peak tan δ values, were used to determine the time to gelation of the resin. Sample shrinkage was measured by measuring the distance between the plates at the same sampling rate as the other metrics.

Graph ‘d’ of FIG. 6 shows the storage modulus for resin compositions comprising 1:1 Reactive Diluent (1, 2, 3): Prepolymer 1, and a Control, which did not contain a reactive diluent. Graph ‘e’ of FIG. 6 shows the peak tan δ values for each of the resin compositions comprising 1:1 Reactive Diluent (1, 2, 3): Prepolymer 1, and a Control, which did not contain a reactive diluent. Graph ‘f’ of FIG. 4 shows the time to gelation of each of the resin compositions comprising 1:1 Reactive Diluent (1, 2, 3): Prepolymer 1, and a Control, which did not contain a reactive diluent.

Resin compositions containing Reactive Diluents 1 and 2 are Comparative Examples, whereas resin compositions containing Reactive Diluent 3 are Examples of the invention.

Referring also to FIG. 7, there is shown graph ‘j’ and graph ‘k’. Graph ‘j’ shows the loss modulus for each resin composition comprising 1:1 Reactive Diluent (1, 2, 3): Prepolymer 1, and a Control, which did not contain a reactive diluent. Graph ‘k’ shows the normalized shrinkage of the gelled resin compositions comprising 1:1 Reactive Diluent (RD1, RD2, RD3): Prepolymer 1, and a Control, which did not contain a reactive diluent.

The control resin composition displayed the most rapid curing (graph ‘f’) and the greatest storage moduli (graph ‘d’), indicating the formation of a solid polymer gel inside 5 seconds of exposure.

Similar response behaviour was found for all resin compositions comprising a Reactive Diluent (1, 2, 3) as determined from the time to the loss factor (tan δ) phase transition maxima (graph e), with varied magnitude of the obtain moduli. This indicates that rapid thiol-ene crosslinking is occurring upon exposure and excitation of the initiator, but prolonged exposure does not continue to crosslink the network substantially, as indicated by the plateau of the curves during this period.

Resin composition containing Reactive Diluent 3 (RD3) displayed a final E′ of approximately 3 MPa, compared to the resin compositions containing Reactive Diluent 1 or 2, which displayed E′ values two orders of magnitude higher, with steady-state values achieved within 5 seconds compared to the 50 s for the resin composition containing Reactive Diluent 3.

Shrinkage of the cured resin was examined during the same crosslinking experiments, with the distance between the plates quantified to determine shrinkage as a result of photocuring. The variation of shrinkage in the without initiator was found to be less than 1%, with the change in gap most likely due to slight evaporation or reordering of the polymer resin. The cured resin composition containing Reactive Diluent 3 and the cured resin composition of the Control displayed shrinkages of less than 3%, as did the cured resin composition containing Reactive Diluent 3. However, the cured resin composition containing Reactive Diluent 2 displayed a final shrinkage of 10%, which may indicate contributions of hydrogen bonding during the curing process. All resin compositions had the same polycarbonate molecular weight/dispersity and concentration of thiol crosslinker (pentaerythritol tetrakis(3-mercaptopropionate)), indicating this difference may be due to the network itself rather than a physical change.

Referring now to FIG. 8, there is shown a series thermal sweeps examining storage moduli (graph ‘a’) and loss factors (graph 131 This illustrates thermal analysis of films formed from a cured resin composition comprising Reactive Diluent 3. Graph ‘a’ shows dynamic mechanical analysis heating from −30° C. to 200° C. at 10° C./min with cooling to ambient at 1° C./min comparing storage modulus per cycle.

Thermal sweeps were conducted at 2° C.×min⁻¹, starting at −30° C. and ending at 200° C. before cooling to ambient conditions at an average initial rate of 10° C.×min-1 to 60° C., followed by 2° C.×min⁻¹ to room temperature, as which point the scaffold was cycled again for 15 cycles. 21 The peak ratio between the loss and storage moduli (E″/E′, tan δ) was defined as the Tg. This method was used to determine curing kinetics of the films, as well. Relaxation kinetics studies of the printed scaffolds were conducted using submersion DMA at 37° C. in phosphate buffered saline (PBS) solution. Scaffolds (1 cm 3) were placed in compression and deformed 10 μm, ¹ Hz with a preload of 0.1 N at ambient conditions for approximately 60 s. At this time, the scaffold was then immersed in the PBS solution and held isothermally as the same load was applied for 60 min. Storage moduli and tan δ values were recorded as a function of time to determine the behaviour of the polymer during initial submersion/introduction to biologically-mimicking conditions.

Rectangular dynamic mechanical analysis was performed (DMA; Mettler-Toledo TT-DMA system (Mettler-Toledo A G, Schwerzenbach, Switzerland)). Samples were prepared via 3D printing sample bars (2.0 cm×0.5 cm×0.2 cm). Samples were analyzed in tension mode using autotension mode, with a frequency of ¹ Hz, a preload force of 1.0 N, and a static force of 0.1 N. The measurements were analyzed using Mettler-Toledo STARe v.10.00 software. Three samples were used in each analysis.

Graph b shows the loss factor (tan δ) peak per cycle. Graph ‘c’ shows the peak value as a function of cycle number. Graph ‘d’ shows the storage moduli and loss factor for films formed from cured resin compositions comprising 1:1 ratios of Reactive Diluent (1, 2, 3) to Prepolymer 1.

Referring also to FIG. 9, there is shown Arrhenius approximations with a scaling factor used to determine the number of days at 20° C. as a result of thermal curing (a), the equivalent number of days at elevated temperature (b), and the number of days at 150° C. as a result of the thermal cure cycle sweeps conducted (c).

The results shown in FIG. 8 were compared with those shown in FIG. 9, to determine the time and temperature at which to cure a part formed from a resin composition according to the invention, equating to an average cure temperature of ˜150° C. (assuming constant heating and cooling rates, maintained through liquid nitrogen vapor flow through the cure chamber).

After 13 thermal cycles, the polymeric films do not display a different T_{g} (graph c). Mechanical evaluations (storage and complex moduli, graph a and graph b) indicate a distinct change after the initial cure (nearly an order of magnitude in the glassy polymer storage modulus at 20° C. below the T_{g}), after which only minimal changes are found. This behaviour is in line with qualitative evaluations of printed monoliths when examined by hand. Based upon an Arrhenius relationship with a conservative 1.5 aging factor utilized, as is standard for medical material aging studies, this indicates that a single cycle (52 min) can be used to mature the green strength of the polymer to the same degree as 1 day at ambient conditions (or ˜25 min at 120° C., a typical curing temperature), and that 13 cycles is the equivalent of 686 days at ambient conditions.

Graph 8d shows thermal sweeps of cured resin compositions comprising Reactive Diluents 1, 2, 3 respectively in a 1:1 ratio with Prepolymer 1. This demonstrated shifting T_{g} from polymers containing Reactive Diluent 1 to polymers containing Reactive Diluent 2, with the T_{g} of the polymer shifting by approximately 20° C. or more when examined by Dynamic Mechanical Analysis using the method described above.

Interestingly, the cured resin containing Reactive Diluent 3 displayed a T_{g} that more closely aligns to the behaviour exhibited by cured resin containing Reactive Diluent 2, but was statistically below it, indicating that while modification from an oxygen atom to a nitrogen atom in the urethane linkage (Reactive Diluent 1) and urea linkage (Reactive Diluent 2) increases the polymer chain rigidity, the hydrogen bonding associated with the urea linkage also plays a significant and separate role in the material properties, similar to how the packing in aliphatic and aromatic polyurethanes may impact hard segment formation, as well as the selection of different diisocyanate precursor species utilized for the synthesis.

Referring also to FIG. 10, there is shown representative thermograms measured by DSC (Differential Scanning calorimetry) for 2 mg samples of the resin compositions containing one of Reactive Diluent 1, 2, or 3, scanned at 10° C./min.

Thermal analysis by DSC (Differential Scanning calorimetry) (Mettler Toledo, AG, Schwerzenbach, Switzerland) was conducted on approximately 1 mg samples hermetically sealed in aluminium pans and placed in the thermal cell. Samples were chilled from room temperature to −80° C. to 200° C., cycled twice to obtain three heating cycles. The half-height transition of the pseudo-second order transition of the enthalpy measurement was taken as the T_{g}, with analysis performed in StarAnalysis (Mettler Toledo, AG, Schwerzenbach, Switzerland).

Referring now to FIG. 11, there is shown a graph showing representative stress strain behaviour when examined using tensile loading of cured resin compositions containing the Reactive Diluents 1, 2, and 3 respectively in a 1:1 ratio with Prepolymer 1. The cured resin compositions were ASTM D638 Type IV printed dogbones tested at 5 mm/min at ambient temperature and humidity wherein (n=7). These were examined using uniaxial tensile testing (Testometric MCT-350, 100 kgf load cell, Testometric Company Ltd, Rochdale, United Kingdom) at ambient moisture and temperature. Samples were placed in the tension clamps and allowed to vibrationally equilibrate for 600 s, at which point each sample was extended at 5 mm/min until failure. Seven samples were run per composition.

It is shown that the cured resin compositions containing Reactive Diluent 1 display a more elastomeric response, with a lower Young's modulus (0.98 MPa) but strains at failure of approximately 100% without a discernible yield point. Conversely, cured resin compositions containing Reactive Diluent 2 display moduli of 35.5 MPa and ultimate stresses of up to 21.6 MPa. However, strain to failure is limited to approximately 60% for these materials, which while still several times superior to commercially available resins, this is nearly half of that of the cured resin compositions containing Reactive Diluent 1.

The cured resin compositions containing Reactive Diluent 3 display attributes of both materials, with strain to failure of nearly 120% and ultimate stresses (28.2 MPa) greater than that of the Urea materials. However, the elastic modulus (15.7 MPa) is lower than that of the Urea materials, an expected trade off when obtaining a tougher material.

Without wishing or intending to be bound by any particular theory, we believe that this may indicate the roles that the hydrogen bonding may play in the material properties. The more rigid urea bond (e.g. of Reactive Diluent 3), without the oxygen atom, produces a more rigid polymer chain that is observed with greater mechanical strength, but the lack of hydrogen bonding allows for more chain slippage, resulting in higher strains prior to rupture of the bulk. This may explain why the cured resin compositions comprising Reactive Diluent 1, despite the more flexible chain due to the oxygen, display lower stains before failure compared to the cured resin compositions comprising Reactive Diluent 3.

Referring now to FIG. 12, there is shown a graph showing the relaxation kinetics displaying the change in storage modulus (E′) and tan δ of printed scaffolds immersed in PBS at 37° C.

Immersion kinetics of a material provide information about the relaxation behaviour of the polymer when exposed to a specific environment; in the case of biomedically-intended shape memory polymers, the infiltration of solvent may impact the shape recovery behaviours in unexpected ways due to polymer chain plasticization.

Scaffolds fabricated from resin compositions comprising Reactive Diluents 1, 2, and 3 respectively were fabricated and used in this study. The role of hydrogen bonding was explored. Reactive Diluent 2 (comprising a carbamide or urea linkage) has twice the number of nitrogen-based hydrogen bond donors as Reactive Diluent 1 (comprising a carbamate or urethane linkage). Polymers comprising Reactive Diluent 3 should contribute very little hydrogen bonding.

The relaxation of the scaffolds was studied by immersing in PBS to provide insight into how hydrogen bonds are interacting as solvent penetrates the polymer network; this was repeated with deionised water to confirm that the salt presence is not the main driving factor. It is shown that the scaffold fabricated from a resin composition comprising Reactive Diluent 1 (carbamate or urethane linkage) undergoes rapid relaxation as demonstrated by the loss factor (tan δ) without any distinct peak, a sign that the material has already passed through its thermal phase transition region. This interpretation is supported by the lack of shape/strain fixation during shape memory examinations, where polymer chains which are already in their maximum entropic conformations (have already undergone thermal phase transitions) are unable to maintain an applied strain.

By contrast, the resin composition comprising Reactive Diluent 2 (carbamide or urea linkages) displays a small loss factor transition and distinct tail over the immersion time. The resin composition comprising Reactive Diluent 3 (cyclic urea linkage) displays a much greater transition and longer transition time. Normalized loss factors indicate these materials relax for almost the entire length of the study, while the materials containing Reactive Diluent 2 are finished relaxing within approximately 2 minutes. However, the comparison of the shape memory behaviours indicates that this relaxation maximum may be more dependent upon with the polymer chain than intramolecular hydrogen bonding, as the shape fixation of the materials containing Reactive Diluent 3 (cyclic urea linkage) is more in line with the materials containing Reactive Diluent 1 (urethane linkage) compared with the materials containing Reactive Diluent 2 (urea linkage).

Qualitatively, the “relaxed” materials containing Reactive Diluent 2 (urea linkage) are much more rigid compared to the Reactive Diluent 1 (urethane linkage) and Reactive Diluent 3 (cyclic urea linkage), which are softer after immersion.

Referring now to FIG. 13, there is shown porous tissue scaffolds printed by stereolithography, displaying a highly open-pore structure and a low density (a), and the corresponding CT scan of the scaffold from a side view (b). The porous tissue structure is representative of cured resin compositions containing Prepolymer 1 and Reactive Diluent 1, 2, and 3 respectively.

The resin compositions were added in 100 mL quantities to the resin tray, allowing for complete and even coverage of the optical window and the surface of the printing plate. Porous scaffolds were printed by applying the photomask (MiiCraft 50x, BURMS, Jena, Germany) and corresponding irradiation to the 50 μm thick slice at 10 s intervals, using 405 nm light. Base plates were burned in from Prepolymer 1 resin at 75 s, with four layers to secure the print; per slice time was varied by photoinhibitor concentration, however approximately 10 s was typically sufficient without overcuring. Post-printing, samples were cut from the plate and immersed in acetone for approximately 1 h to remove residual resin ink. Other printed monoliths were printed with slight variations in printing conditions. After the cleaning with acetone, printed samples were allowed to dry overnight at ambient conditions.

Printing resulted in rapid layer solidification and minimal shrinkage of the porous prototype. Resin compositions containing Prepolymer 1 and Reactive Diluent 1 or Reactive Diluent 2 required a photoinhibitor (Paprika Extract) to ensure accurate resolution balanced with rapid photocuring (10 s per 50 μm slice). However, the resin composition comprising Reactive Diluent 3 did not require an additional photoinhibition agent to ensure accurate feature resolution. This was efficient enough to require 60 s per 50 μm slice at 1:2 ratio. It should be noted that at 1:1 ratio of Prepolymer 1 with Reactive Diluent 3, the resin was incapable of solidifying into solid parts during DLP (digital light processing), even at 3 wt. % photoinitiator, and ultimately could only be solidified using 405 nm light at elevated temperatures.

Referring now to FIG. 14, there is shown a series of graphs (a, b, c) showing the shape memory behaviour of printed porous scaffolds formed from resin compositions comprising one of Reactive Diluents 1, 2, and 3 respectively. Graph d shows the strain recoveries/fixation as functions of temperature in 2D.

Dynamic mechanical analysis (DMA) shape memory experiments were performed using porous scaffolds in compression mode. The samples were equilibrated at 60° C., deformed by ˜30% (load dependent deformation) and cooled to −20° C. Once the sample was isothermal with the cooled chamber, the load was removed and the sample expansion was monitored as a function of force and displacement of the compression clamp as the sample was heated to 60° C. at 10° C.×min⁻¹.

The printed scaffolds in each case display an initial regime of rapid strain recovery, up to at least 40%. This seems to be elastic contributions of the macroscopic structure rather than being solely a factor of the polymer, and is qualitatively found during bulk compression and shape fixation of the scaffolds, as well. After this initial recovery, the printed scaffolds comprising Reactive Diluent 2 (urea linkage) display consistent strain fixation up to 35° C., at which point shape recovery begins. The rate of recovery is also different compared with the printed scaffolds comprising Reactive Diluent 1 or 3, possibly due to the hydrogen bonding density. The material of the printed scaffold comprising Reactive Diluent 2 possess twice as many possible hydrogen bonding sites, which would both increase the amount of energy needed to produce shape recovery (hence the increased temperature) but the rate at which these temporary bonds are interrupted should be lower simply due to their relative density. Without wishing or intending to be bound by any particular theory, we believe that the high crosslink density is sufficient to disrupt any possible crystalline domains, resulting in only a rigid polymer backbone (as seen in materials containing Reactive Diluent 2 with a urea linkage) possessing stronger intermolecular interactions relative to the materials containing Reactive Diluent 1 (urethane linkages) or Reactive Diluent 3 (cyclic urea linkages).

Advantageously, the resin composition according to the invention comprises a photoinhibitive compound that provides spatial printing control by preventing light penetration from the stereolithography light source during printing of a 3D object. This enables finely resolved surface features to be printed, which allows complex objects to be printed efficiently and economically using light.

More advantageously, the photoinhibitive compound is incorporated into the crosslinked polymer matrix, which prevents leaching of compounds that are unbound to the polymer.

Even more advantageously, the properties of the crosslinked polymer fabricated from the resin composition may be tuned by adjusting the amount and type of each component. The use of different quantities of prepolymer and/or reactive diluent and/or crosslinker in the resin composition enables the thermomechanical properties of the resulting crosslinked polymer to be tuned. For example, the elasticity or rigidity of the crosslinked polymer may be tuned by adjusting the amount of prepolymer and/or reactive diluent and/or crosslinker in the resin composition. Without wishing to be bound by any particular theory, it is thought that the degree of crosslinking provides this effect. However, it is additionally believed that the thermomechanical properties may be tuned by adjusting the hydrogen bonding density. It has been found that the use of reactive diluents comprising a urea, e.g. a cyclic urea as found in Reactive Diluent 3 provides a polymer with comparatively less hydrogen bonding than those polymers fabricated using Reactive Diluents 1 or 2. It has been shown that a reduction in hydrogen bonding density is associated with polymers having a higher strain to failure, and higher ultimate strength without significant failure or fracturing regions. Adjustment of the hydrogen bonding density may also be used to tune shape memory properties because this is important for shape fixation. Relaxation of the polymer chains in hydrated environments is found to be dependent on hydrogen bonding; polymers without H-bonding or with reduced H-bonding undergo significant, rapid relaxation (displayed as both mechanical responses as well as thermal phase transitions of the materials).

Properties that may be tuned by adjusting different amounts and chemical structures of one or more of prepolymer and/or reactive diluent and/or crosslinker include degradability, shape memory properties, processability, and thermomechanical properties such as glass transition temperature.

Moreover, the crosslinked polymer may be functionalised post-polymerisation using the methods described in WO2018/229456A1.

It will be appreciated by those skilled in the art that any number of combinations of the aforementioned features and/or those shown in the appended drawings provide clear advantages over the prior art and are therefore within the scope of the invention described herein.

1.-30. (canceled)

31. A resin composition, the resin composition comprising a prepolymer, a reactive diluent, and optionally a crosslinker, wherein the reactive diluent has an absorption maximum (κₘₐₓ) or excitation maximum (λₘₐₓ) of between 350 and 520 nm for example in acetone and at pH 7.

32. A resin composition according to claim 31, wherein the reactive diluent comprises an O═C—N linkage, which is selected from a carbamate linkage or a carbamide linkage.

33. A resin composition according to claim 31, wherein the reactive diluent comprises at least one cyclic urea moiety.

34. A resin composition according to claim 31, wherein the reactive diluent has a relative molecular mass (Mᵣ) selected from:
i. less than 1000;
ii. 200 or more;
iii. 200 or more and less than 1000.

35. A resin composition according to claim 31, wherein the reactive diluent comprises at least one unsaturated side chain.

36. A resin composition according to claim 31, wherein the reactive diluent comprises the general formula (i): 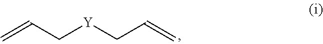
wherein Y comprises one or more cyclic moieties of general formula (iii):

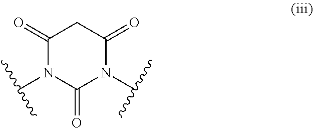

37. A resin composition according to claim 36, wherein Y comprises one or more alkyl or aryl groups selected from unsubstituted alkyl or aryl groups or substituted alkyl or aryl groups.

38. A resin composition according to claim 37, wherein the one or more alky; or aryl groups bridge cyclic moieties of general formula (iii).

39. A resin composition according to claim 31, wherein the prepolymer comprises repeating units, each repeating unit having at least one unsaturated side chain.

40. A resin composition according to claim 31, wherein the prepolymer comprises repeating units, each repeating unit having at least one carbonate linkage.

41. A resin composition according to claim 40, wherein the prepolymer is of the following general formulae: 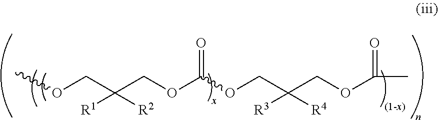
wherein R¹ is an aliphatic or an aromatic moiety or group, R² is an aliphatic or an aromatic moiety or group, R³ is an aliphatic or an aromatic moiety or group, and R⁴ is an aliphatic or an aromatic moiety or group, and wherein n is a number that is less than one hundred and x is a number between 0 and 1.

42. A resin composition according to claim 31, further comprising one or both of a viscosity modifier and a photoinitiator.

43. A resin composition according to claim 31, wherein the prepolymer is present in a quantity of between 10 and less than 100 w/w % of the total composition, the total quantity of reactive diluent is present in a quantity of between greater than 0 and 50 w/w % of the total composition, and a crosslinker is present in a quantity of between 0 and 50 w/w % of the total composition, and optionally wherein the initiator is present in a quantity of between 0 and 5 w/w % of the total composition.

44. A resin composition according to claim 31, comprising a cross linker, wherein the crosslinker comprises a moiety that is capable of reacting with at least one unsaturated side-chain of the prepolymer and/or the reactive diluent.

45. A resin composition according to claim 44, wherein the crosslinker comprises at least two thiol moieties.

46. A cross-linked polymer comprising a resin composition of claim 31 which has been cross linked and wherein the cross-linked polymer is a shape memory material that is responsive to a temperature change to effect a shape change.

47. A cross-linked polymer according to claim 46, wherein the cross-linked polymer is degradable into degradation products upon exposure to one or more of water, acid, base, heat, enzymes, solvent, and/or oxidisers.

48. A device comprising the cross-linked polymer of claim 46, wherein the device is an implantable medical device comprising pore sizes in the range from approximately 200 μm to 1500 μm.

49. A method of fabricating a cross-linked polymer, the method comprising:
i. providing a resin composition according to claim 31;
ii. optionally, contacting the resin composition with a initiator;
iii. optionally, providing an energy source to activate the initiator.

50. A method according to claim 49, further comprising step vi. providing a reagent for functionalising an at least one unsaturated side chain with a moiety selected from:
a. An alkylating moiety;
b. hydrophobic moiety;
c. a cell adhesion moiety.
